(19) United States
(12) Patent Application Publication
(10) Pub. No.: US 2018/0148679 A1
(43) Pub. Date: May 31, 2018

(54) Liquefaction of cellulose-containing feedstocks

(71) Applicant: Purdue Research Foundation, West Lafayette, IN (US)

(72) Inventors: Michael Ralph Ladisch, West Lafayette, IN (US); Eduardo De Aquino Ximenes, West Lafayette, IN (US); Daehwan Kim, West Lafayette, IN (US); Thomas Richard Kreke, West Lafayette, IN (US)

(73) Assignee: Purdue Research Foundation, West Lafayette, IN (US)

(21) Appl. No.: 15/570,413

(22) PCT Filed: Apr. 29, 2016
(86) PCT No.: PCT/US2016/030195

§ 371 Date: Oct. 30, 2017

Related U.S. Application Data
(60) Provisional application No. 62/154,403, filed on Apr. 29, 2015.

(51) Int. Cl. C12M 1/00; C12P 21/06; C12P 19/14; C12P 7/10; C12P 7/64; C12P 19/02

(52) U.S. Cl. CPC C12M 45/09; C12P 21/06; C12P 19/02; C12P 7/10; C12P 7/6418; C12P 19/14

(57) ABSTRACT

A method for producing a pumpable slurry from a cellulose-containing feedstock is presented herein.

### CROSS-REFERENCE TO RELATED APPLICATIONS

The present patent application is related to and claims the priority benefit of U.S. Provisional Patent Application Ser. No. 62/154,403, filed Apr. 29, 2015, the contents of which is hereby incorporated by reference in its entirety into this disclosure.

### TECHNICAL FIELD

The present disclosure generally relates to cellulosic ethanol conversion, and in particular to liquefaction of cellulose-containing feedstocks.

### BACKGROUND

This section introduces aspects that may help facilitate a better understanding of the disclosure. Accordingly, these statements are to be read in this light and are not to be understood as admissions about what is or is not prior art.

The production of cellulosic ethanol from agricultural residues commonly utilizes corn stover as a feedstock in the process of cellulose pretreatment, enzyme hydrolysis, and fermentation. Plants utilizing such processes combine the ethanol conversion and recovery processes. The sequence of processing steps in a corn dry grind facility is analogous to where pretreatment includes cooking and enzyme treatment in order to liquefy the corn and achieve a pumpable slurry. However, unlike cellulose pretreatment, where solid materials are cooked in water or dilute acid at 180 to 200° C., the cooking step in a corn facility is carried out at 110° C. using a liquid slurry. Consequently, due to the higher temperatures and pressures as well as the need to move solids through the process, cellulose pretreatment equipment costs are 5 to 10 times higher when compared to pretreatment equipment used for corn. Given also that production of cellulosic ethanol is beginning to increase, it would be highly beneficial to have a process that aims to liquefy cellulosic biomass feedstocks before pretreatment in order to achieve pumpable slurries of lignocellulosic feedstocks, and to reduce temperatures and pressures required for pretreatment.

### SUMMARY

In one aspect, a method for producing a pumpable slurry from a cellulose-containing feedstock is presented, which includes utilizing at least one of cellulase and xylanase enzymes. In another aspect, a method for producing a pumpable slurry from a cellulose-containing feedstock is presented. The method can include liquefying a cellulose-containing feedstock into a pumpable slurry, wherein the liquefying of the cellulose-containing feedstock is carried out using an enzyme in an agitated vessel. In an embodiment, the methods can include an initial average particle length in the range of about 20 microns to about 10 cm. In another embodiment, the methods can include an initial average particle length in the range of about 0.1 mm to about 10 mm. In yet another embodiment, the methods can include temperatures between 4° C. and 100° C. and a pressure of 1 bar. In yet another embodiment, the methods can include temperatures above 100° C. In yet another embodiment, the methods can include temperatures between 4° C. and a temperature above the critical point. In yet another embodiment, the methods can include temperatures between 4° C. and 260° C. In yet another embodiment, the methods can include the pressure is above the pressure at which water vaporizes. The enzyme can include any one of or a combination of cellulose, xylanase, protease, β-xylosidase, β-glucosidase, and/or lipase.

In yet another embodiment, the methods can be carried out in a fed-batch addition of the cellulose-containing feedstock. In yet another embodiment, the methods can be carried out by continuously-fed addition of cellulose-containing feedstock. In yet another embodiment, the methods can result in a slurry concentration of at least 300 g/L. Further, in yet another embodiment, the methods can result in a slurry concentration of at least 400 g/L. In yet another embodiment, the methods can result in a slurry concentration of at least 500 g/L, and in yet another embodiment, the methods can result in a slurry concentration of at least 300 g/L. In yet another embodiment, the methods can result in a slurry concentration of between about 200 g/L to about 220 g/L.

The cellulose-containing feedstock can be any one of or a combination of soybean hulls, corn fiber, corn cobs, sugarcane bagasse, wheat straw, hardwood, poplar, energy crops, corn, soybean residues, and/or switch grass.

### BRIEF DESCRIPTION OF FIGURES

FIG. 1 is a process diagram showing unit operations for a cellulose to ethanol fermentation and biorefinery facility.

FIG. 2A is a photograph showing dried pericarp.

FIG. 2B is a photograph showing untreated pericarp.

FIG. 2C is a photograph showing pericarp liquefied by Multifect pectinase.

FIG. 2D is a photograph showing pericarp added to Depol 692.

FIG. 3A is a photograph showing an embodiment of the reactors for liquefaction.

FIG. 3B is a photograph showing soybean hulls after 24 hours of mixing with no enzyme (“Control”) and after 25 hours of mixing with the liquefied hulls in Multifect+Spezyme CP enzymes (“Liquefied”).

### DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

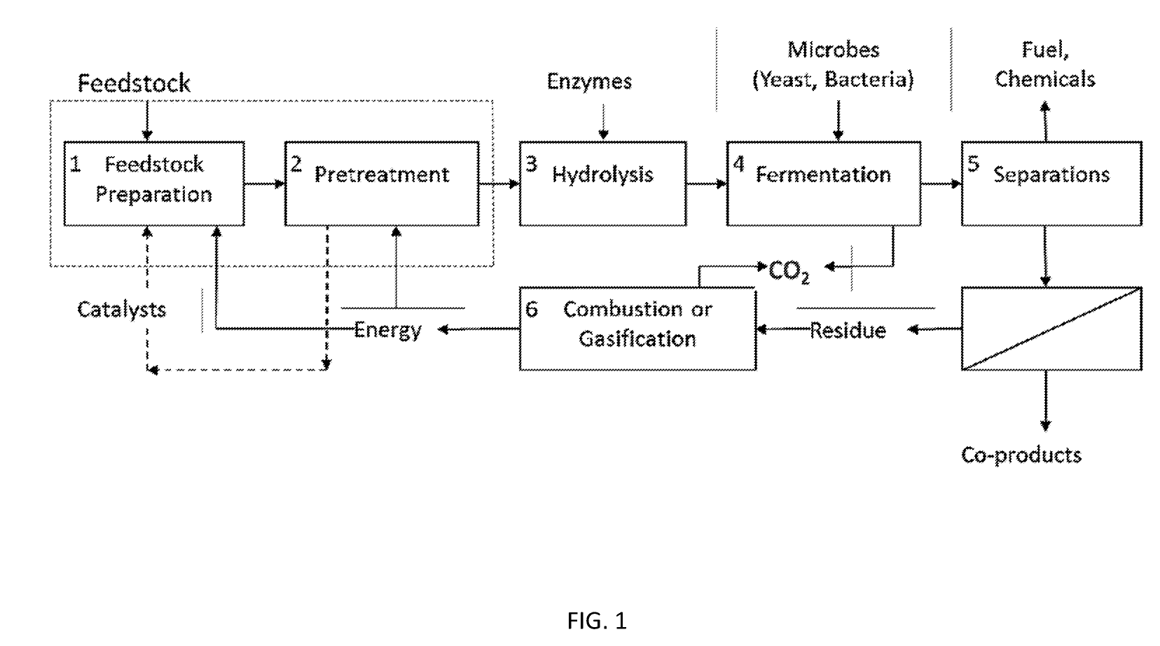

A method is herein presented to respond to the unmet need for a process that aims to liquefy cellulosic biomass feedstocks before pretreatment (FIG. 1, step 2) in order to achieve pumpable slurries of lignocellulosic feedstocks, and to reduce temperatures and pressures required for pretreatment. Such a process can simplify equipment requirements for cellulose pretreatment and fermentation and can have a major impact on introducing cellulose conversion to existing corn-to-ethanol dry grind facilities. In turn, this can lead to demand for sustainable sources of cellulose-containing feedstocks such as corn fiber, soybean hulls, and corn and soybean residues from corn and soybean farmers.

Liquefaction results in a shear thinning fluid, and energy can be required to begin the pumping process. A pumpable fluid may require that the apparent viscosity be less than 1000 Pa/sec at a shear rate of 0.1 l/sec or 0.3 pa/sec at a shear rate of 100 l/sec. The viscosity of the pumpable fluid can be lower, with water having a dynamic viscosity of 1 mPa per second at 20 C. (1 Pascal=1000 mPa). For purposes of this disclosure, the variation in shear rate within this range can define a pumpable slurry, (i.e., a slurry that can flow as a fluid through a pipe or pump). Pumps may include but are not restricted to a centrifugal pump, progressive cavity pump, and/or a peristaltic (tube pump) pump. Because the flow of such a slurry and properties of the slurry at a microscopic level are complex and involve interactions between particles or sheets of particles, the term apparent viscosity is used. The apparent liquid viscosity is a function of temperature, with viscosity decreasing with increasing temperature. It is also apparent to one skilled in the art that dilution of the slurry with water or other aqueous fluid will ultimately result in a pumpable slurry, by virtue of the dilution effect. It is useful obtain a concentration of solids that exceeds 3 weight % in order to achieve an economic equipment size for processing a pre-determined amount of dry matter per hour. Liquefaction of the of the soybean hulls and other cellulosic soybean residues at concentrations that range between about 3 and about 30% may resemble a paste before treatment with enzyme while providing a fluid with fluid properties after treatment described herein.

In one aspect, a method for producing a pumpable slurry from a cellulose-containing feedstock is presented, which includes utilizing at least one of cellulase and xylanase enzymes. In another aspect, a method for producing a pumpable slurry from a cellulose-containing feedstock is presented. The method can include liquefying a cellulose-containing feedstock into a pumpable slurry, wherein the liquefying of the cellulose-containing feedstock is carried out using an enzyme in an agitated vessel. In an embodiment, the methods can include an initial average particle length in the range of about 20 microns to about 10 cm. In another embodiment, the methods can include an initial average particle length in the range of about 0.1 mm to about 10 mm. In yet another embodiment, the methods can include temperatures between 4° C. and 100° C. and a pressure of 1 bar. In yet another embodiment, the methods can include temperatures above 100° C. In yet another embodiment, the methods can include temperatures between 4° C. and a temperature above the critical point. In yet another embodiment, the methods can include temperatures between 4° C. and 260° C. In yet another embodiment, the methods can include the pressure is above the pressure at which water vaporizes. The enzyme can include any one of or a combination of cellulose, xylanase, protease, β-xylosidase, β-glucosidase, and/or lipase.

In yet another embodiment, the methods can be carried out in a fed-batch addition of the cellulose-containing feedstock. In yet another embodiment, the methods can be carried out by continuously-fed addition of cellulose-containing feedstock.

The cellulose-containing feedstock can be any one of or a combination of soybean hulls, corn fiber, corn cobs, sugarcane bagasse, wheat straw, hardwood, poplar, energy crops, corn, soybean residues, and/or switch grass. In yet another embodiment, the methods can result in a slurry concentration of at least 300 g/L. In yet another embodiment, the methods can result in a slurry concentration of at least 400 g/L. In yet another embodiment, the methods can result in a slurry concentration of at least 500 g/L. In yet another embodiment, the methods can result in a slurry concentration of at least 300 g/L. In yet another embodiment, the methods can result in a slurry concentration of between about 200 g/L to about 220 g/L.

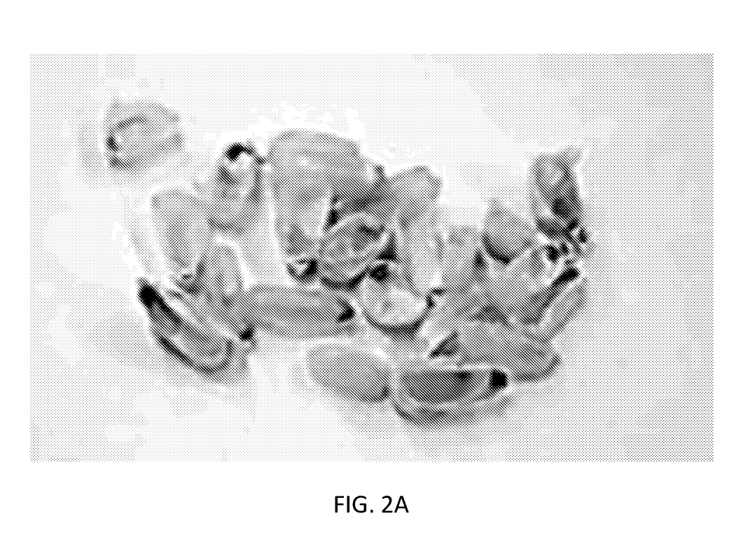

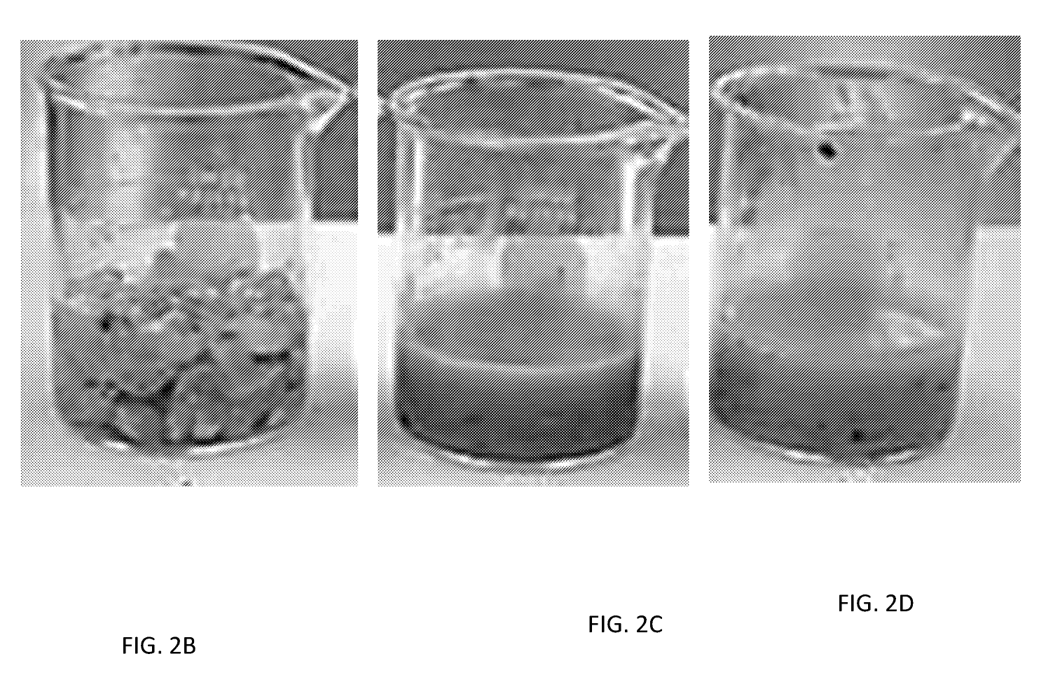

As mentioned above, and as demonstrative of the principles set forth herein, in an embodiment of the present disclosure, soybean hulls are utilized as cellulose-containing feedstock. As demonstrated herein, flow properties of the cellulose-containing pericarp can be modified prior to pretreatment by using cellulase and xylanase enzymes to liquefy pericarp into a slurry at a concentration of 300 g of dry pericarp/L, an example of which is illustrated in FIGS. 2A and 2B, 2C, and 2D (which are photographs depicting liquefaction before pretreatment; FIG. 2A depicts dried pericarp; FIG. 2B depicts untreated pericarp; FIG. 2C depicts pericarp liquefied by Multifect pectinase; FIG. 2D depicts pericarp added to Depol 692; these pictures were taken after 72 hours of mixing at 50° C. with 250 IU enzyme/gram dry pericarp). An optimized enzyme formulation and bioreactor configuration enables us to similarly liquefy soybean hulls for producing cellulose ethanol. As an aside, a cost-saving aspect of such a process involves the observation that hulls that are already collected at soy processing plants can be shipped to nearby corn to ethanol plants, liquefied, and converted to ethanol using cellulose-hydrolyzing enzymes and yeast in existing dry-grind facilities. Experimental results using soybean hulls show liquefaction to at least 300 g/L and currently up to 500 g/L is possible (see FIGS. 2A and 2B1 and 2B2, which are photographs depicting the liquefaction of soybean hulls before pretreatment.

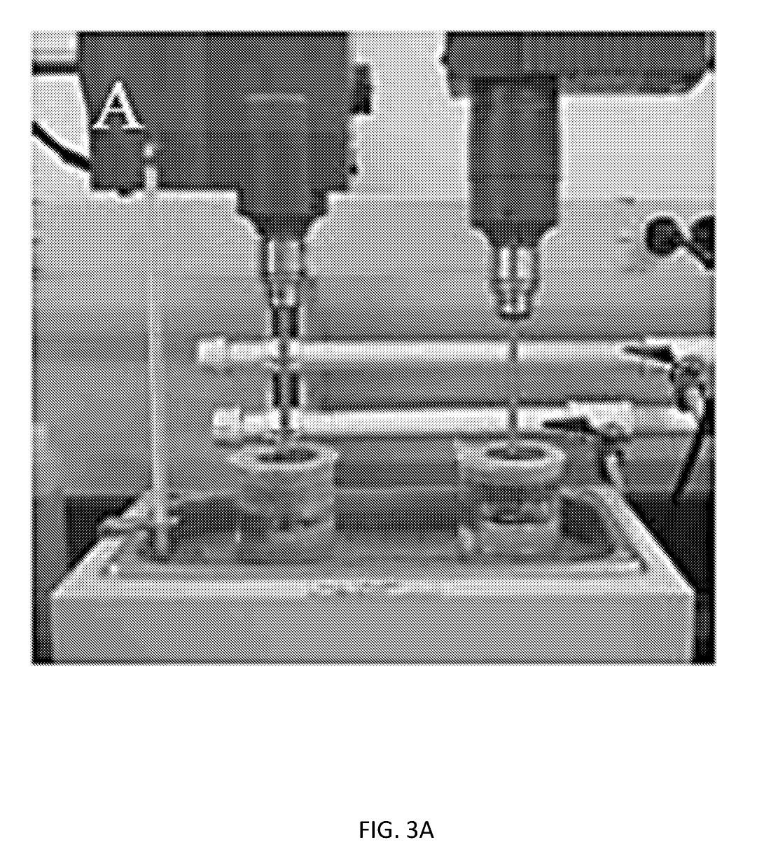

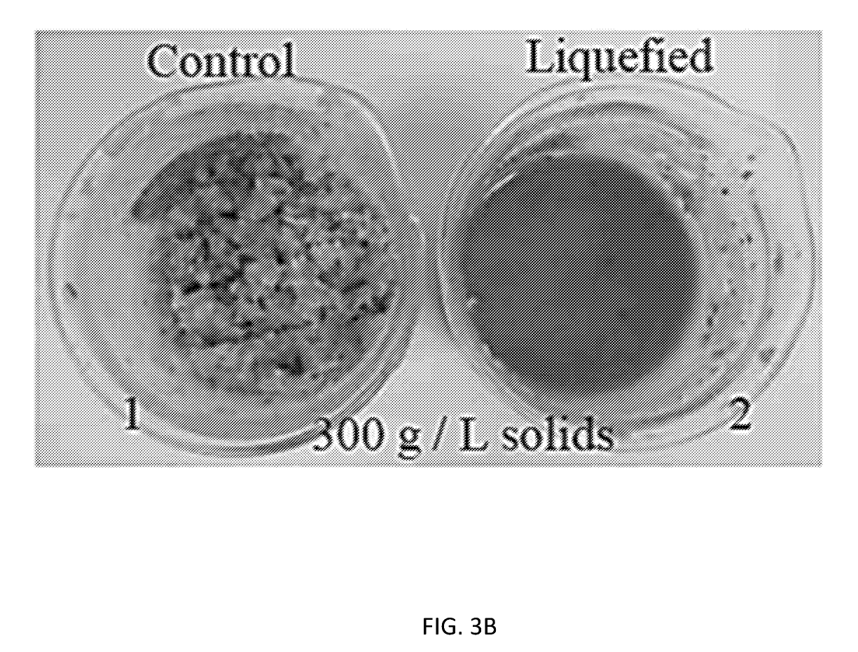

Similarly, in the case of other feedstock, for example corn stover, 200-220 g/L can be achieved; switch grass is another example of a feedstock. Referring back to the example of soybean hulls, FIG. 3A is a photograph showing an embodiment of the reactors for liquefaction; FIG. 3B is a photograph of the soybean hulls after 24 hours of mixing with no enzyme (i.e., the control); and a photograph of the soybean hulls after 24 hours of mixing with the liquefied hulls in Multifect+Spezyme CP enzymes). Liquefaction was carried out through fed-batch addition of soybean hulls (that had not been roasted) to commercially available enzymes as described further below.

In yet another embodiment, the herein described process can utilize enzymes to liquefy soybean hulls into a pumpable slurry for the purpose of producing cellulosic ethanol and protein-enhanced animal feed additives in existing corn dry grind facilities. In yet another embodiment, which may include liquefaction of corn pericarp, a pumpable slurry containing 300 g/L solids can be obtained. Enzyme formulations and conditions can be defined that lie within the operational parameters of existing corn to ethanol processes (including temperatures, pressures, pH, residence times, water balance, separations). Similarly, enzyme formulations and conditions for any of the herein described cellulose-containing feedstocks can be defined according to the principles described herein. In one embodiment, temperatures may be between 4° C. and 100° C. at 1 bar. In another embodiment, the temperature may be above 100° C. where the system pressure is above the pressure at which water vaporizes, and can result in a pumpable fluid that is suitable for, for example, a pretreatment reactor at 200° C. In another embodiment, the temperatures may be between about 4° C. and about 260° C. The results of such processes add value to soybean hulls (and corn pericarp) by such cellulose-containing solid materials becoming amenable to processing in existing corn ethanol plants by liquefying these materials at concentrations of 300 g/L or higher.

The herein described methods can benefit growers (for example soybean growers) by providing an additional value-add market for the particular cellulose-containing feedstock being used (for example, soybean hulls, and potentially soybean field residues). In the case of soybean hulls, the soybean hulls are already collected in soybean crushing facilities, and are currently used as animal roughage. This approach therefore enables existing corn to ethanol plants to utilize cellulose from an identifiable source to produce ethanol and provide a value-add use for soybean hulls. Since soybean hulls are typically collected at soybean crushing plants that are geographically located close to corn dry-grind facilities, a feedstock supply chain for this cellulosic material is already established.

Additional uses can include enhancing demand for corn and soybeans by expanding the product portfolio and revenue of current dry grind corn to ethanol facilities as well as providing another value-added market for soybean hulls. As mentioned above, other cellulose-containing feedstocks can be used, which include but are not limited to any one of or a combination of soybean (field) residue, corn stover, cobs and fiber, wheat straw, sugarcane bagasse, hardwood (poplar) and purposely grown energy crops in corn dry-grind plants. For these cellulose-containing feedstocks to be obtained, the harvest, collection and supply need to take conservation tillage practices into consideration (see for example, Eck and Brown, 2004). In yet other embodiments, similar considerations apply if a portion of the soybean residues (or other cellulose-containing feedstocks) are collected from the field.

In yet another embodiment, and as a further example of the principles described herein, high yields at reduced enzyme loadings for pretreated hardwood have been demonstrated. The inhibitory effects of lignin and phenolics on enzymes have been defined. However, the major challenges of large capital cost required to construct new cellulosic biorefineries and the high level of enzyme loading required to convert pretreated lignocellulosic materials to sugars, still remain. While fundamental research on enzyme inhibition has identified pathways to reducing inhibition (Ko et al., 2014a, 2014b; Kim, 2013, 2014; Ximenes, 2010, 2011), other areas still need to be addressed. These include the designing, building, and operating cellulose to ethanol plants that are economically attractive.

The methods described herein, in yet another embodiment, utilize existing infrastructure in corn dry mills. Enzymes and/or microorganisms already developed for cellulose conversion can enable the corn-to-ethanol plants to transition to cellulose conversion. This can increase the portfolio of products manufactured in these facilities and to identify and utilize existing, already collected, biomass as a cellulosic feedstock in corn-to-ethanol plants.

As mentioned above, soybean hulls are one type of cellulose-containing feedstock. Biotechnologies and cellulose processing methods are advancing to the point that the challenge of being able to obtain a pumpable slurry of the cellulosic material becomes important so that these technologies may be used in existing corn-to-ethanol plants. Conversion of soybean hulls into a pumpable slurry at concentrations of 400 g/L or higher can enable alcohol concentrations of up to 120 g/L (12% w/v) to be obtained. The hulls can be processed in equipment that can heat, cook, mix, and separate cellulose feedstocks in a manner that is similar to corn slurries. A previously published laboratory study showed that soybean hulls, without liquefaction can be loaded into a fermenter at up to 15% (150 g/L) and then hydrolyzed and fermented to ethanol concentrations of 2.5 to 3% (25 to 30 g/L) over a 9-day period. The remaining solid residue containing 25% protein compared to 10% in the starting material (Mielenz et al., 2009), is recovered. The proposed method improves rates, concentrations and yields by liquefying the hulls before further processing them and obtaining significantly higher ethanol concentration.

A first step in achieving the goal of cellulose conversion in a dry-grind plant is to transform soybean hulls into a pumpable fluid. Data has demonstrated this is possible for soybean hulls, corn residue (i.e., stover), corn fiber (pericarp), and sugarcane bagasse.

Slurries of soybean hulls of 300 g/L or higher at enzyme loadings that enable this result can be obtained in 48 hours or less. Conditions will be selected, developed, and optimized so that they may be scaled-up in equipment that is familiar to operators of a dry grind facility. Liquefaction results in a pumpable fluid, suitable for hydrolysis and fermentation to ethanol, using existing fermentor designs.

Published compositional analysis for soybean hulls (Table 1) show cellulose and hemicellulose contents are similar to corn stover, with a lower lignin content and a higher protein content. However, it is important to note the published analysis only accounts for 80% of the dry matter.

TABLE 1
Composition of Soybean Hulls (Meilenz et al., 2009).

| | |
|---|---|
| Cellulose | 38.4 |
| Hemicellulose | 10.2 |
| Pectin (est.) | 10.0 |
| Protein | 10.7 |
| Lignin | 2.8 |
| Ash | 5.8 |
| Total | 77.9 |

Liquefaction is carried out prior to cellulosic pretreatment, and the slurry itself is pretreated after liquefaction in liquid hot water using conditions previously defined for corn stover and DDG from a dry grind facility (Zeng, 2010; Kim et al., 2008a,b). These data will compare ethanol yields from liquefied hulls, before and after pretreatment, and determine the economic consequences of alternate approaches.

In yet another embodiment, if the liquefied hulls are readily converted to ethanol, without pretreatment, hydrolysis and fermentation conditions can be optimized with the observation that residual cellulose remains, together with the protein, in the post-fermentation residues. In another embodiment, the liquefied slurry is pretreated in liquid hot water and then converted to ethanol, by a combination of enzyme hydrolysis and fermentation. The remaining residue can still be valuable as an animal feed, although will have a different composition than residual liquefied hulls that are processed without pretreatment. The protein content of the hulls also determines the economic value of the hulls.

### Example 1

Materials and Methods:

An example of the materials and a demonstration of the methods disclosed herein are presented below and are based on established protocols for compositional analysis, enzyme hydrolysis, and fermentation (Ko et al., 2014a, 2014b, Kim et al., 2013, 2014, Ximenes et al., 2010, 2011).

Liquefaction of Soybean Hulls:

In one embodiment, the reaction conditions are as follows: One mL of Multifect Pectinase and 1 mL of Spezyme CP are added to 100 mL 0.05M sodium citrate buffer (pH 4.8) in a 250 mL beaker flask capped with a stopper. Soybean hull at 10% moisture is added in 9 g increments at 0, 2, and 4 hours with an additional 6.5 g added at 24 hours, and 11.2 g added at 48 hours until the solids concentration reaches 40% w/v (dry basis) in 100 mL of 0.05M sodium citrate buffer. One mL of Multifect Pectinase and 1 mL of Spezyme CP will be added at 2 hours, and an additional 3 mL of each enzyme to be added at 4 hours. The control run is performed at the same conditions but in the absence of enzyme (same volume of buffer will be added). Agitation will be at 290 rpm in a (New Brunswick Scientific, Innova 44) for 96 hours and at 50° C. Tables 2 and 3 are summaries of a typical run and enzyme loadings. The rates of soybean addition, levels and formulations of liquefying enzymes, and agitation rates are varied with the goal of reducing time, enzyme, and agitation rate while still achieving liquefaction.

TABLE 2
Time points at which materials added.

| | Time (hr) | | | | | |
|---|---|---|---|---|---|---|
| | 0 | 2 | 4 | 24 | 48 | Total |
| Soybean | 9 | 9 | 9 | 6.5 | 11.2 | 44.7 g (40 g |
| hulls (g) | | | | | | dry basis) |
| 0.05M | 100 | — | — | — | — | 100 mL |
| sodium | | | | | | |
| citrate | | | | | | |
| buffer (ph | | | | | | |
| 4.8), (mL) | | | | | | |
| Multifect | 1 | 1 | 3 | — | — | 5 mL |
| Pectinase | | | | | | |
| (mL) | | | | | | |
| Spezyme | 1 | 1 | 3 | — | — | 5 mL |
| CP (mL) | | | | | | |

TABLE 3
Information on enzyme loadings used in EXAMPLE.

| | Mg | Weight of protein | Protein/total weight of |
|---|---|---|---|
| Enzyme | protein/mL | added (mg) | soybean hulls, (mg/g) |
| Multifect | 81.3 | 407 | 9.1 |
| Pectinase | | | |
| Spezyme CP | 82.0 | 410 | 9.2 |

Rheological Measurements:

Viscosity of the slurries are measured at 50° C. shear rates of 0.1 to 1000 in a model AR-G2 rheometer (TA Instruments, USA) using a starch pasting impeller and cup geometry. Viscosity ranges between about 0.01 pascal and 10000 pascals.

Particle Size Measurements.

A Malvern Mastersizer 2000 is used to measure particles between 0.02 to 2000 μm and provides confirmation of the size and distribution of particles that are formed. The Masterziser is based on laser diffraction with the calculation of the particles size is back-calculated from the scattering pattern. For particles in the lower size range, and for particles that may require determination of surface charge, a more appropriate instrument is the Malvern Zetasizer Nano ZS. This instrument measures zeta potential and electrophoretic mobility, as well static light scattering measurement and then relates these to particle size or particle behavior.

Scanning Electron Microscopy.

SEM images of particles in soybean hull slurries sampled at several stages of the liquefaction process are obtained as described previously (Zeng et al., 2012a,b). A JSM-840 Scanning Electron Microscope (JEOL USA Inc., Peabody, Mass.), is used to image either uncoated dried particles of soy hulls at low accelerating voltage or at 5 kV for hull particles sputter coated with AuPd in the presence of argon gas using a Hummer 1 sputter coater (Technics Inc., Alexandria, Va.). The particulates are recovered by sampling the material at times ranging from 0 to 96 hours, drying the slurry sample by lyophilization, and the using an aliquot of the resulting sample for SEM.

Analytical Methods:

Liquefied samples are analyzed for sugar and acetate content using a Bio-Rad Aminex HPX-87H ion exchange column (300 mm×7.8 mm, Bio-Rad Laboratories Inc., Hercules, Calif.) connected to a Milton Roy mini pump (Milton Roy Co., Ivyland, Pa.), Waters™ 717 plus autosampler, and Waters™ 2414 refractive index detector (Waters Corp., Milford, Mass.). The data are stored and processed using Empower™2 Chromatography Data Software (Waters Corp., Milford, Mass.). The mobile phase is 5 mM sulfuric acid in distilled, de-ionized sterile water. The mobile phase flow rate is 0.6 mL/min. The column temperature is maintained at 60° C. (Eppendorf CH-30 Column Heater controlled by an Eppendorf TC-50 (Eppendorf, Westbury, N.Y.)).

The liquefaction characteristics achieved are based on an understanding of the interaction of lignin and lignin-derived inhibitors on cellulose-hydrolyzing enzymes. Results show addition of protein to lignocellulose materials, enables a 5 to 10 times reduction in enzyme loading by blocking adsorption of the enzyme on lignin and redirecting the enzyme to the cellulose components in the cell wall structure. This knowledge provides insights on how to more effectively utilize commercially available enzymes and reduce costs for cellulose conversion. An example is the success in liquefying soybean hulls. While the preliminary data show liquefaction at up to 300 g/L, when this experiment was continued, up to 450 g/L and approaching up to 500 g/L final solids concentration was obtained. Prior liquefaction experiments with corn stover were also successful but required more enzyme, and gave a more viscous material. When a similar approach was tried for corn fiber, a concentration of 300 g/L was readily achieved with a lower apparent viscosity.

The protein content of hulls is significant and that both the hulls and pericarp have a lower lignin content and higher protein content than other forms of lignocellulosic biomass. The unexpected success with soybean hulls is due to the presence of indigenous protein that may block lignin's adsorption/inhibition of the cellulase and hemicellulase enzymes used to liquefy this material. Therefore, value-added products can be obtained from soy and corn residues through processing their cellulosic residues in existing corn ethanol plants, and thereby enhance markets for crops from soybean and corn producers.

### Example 2

Liquefaction processes were carried out with corn pericarp or soybean hull by Multifect pectinase treatment. Initial experiments were performed using corn pericarp, and then analyzed main sugar components like cellobiose, glucose, xylose, arabinose, and acetic acid as an inhibitor (Table 4 and Table 5). The highest glucose conversion of corn pericarp after 48 h, 43.06 g/L, was obtained with other sugars in supernatant. Glucose from soybean hulls was produced after 72 h, 65.21 g/L with a same process. The reduction of glucose has showed that after 48 h or 72 h in corn pericarp and soybean hulls, it may be attributed to the presence of various inhibitors formed during liquefaction process. This result indicates that Multifect pectinase treatment to decompose corn pericarp demonstrates the benefit of hydrolyzing corn pericarp for enhancing cellulose digestibility.

Liquefaction of corn pericarp and soybean hulls to fermentable sugars under the physical-enzymatic condition shows that the herein disclosed methods play a role in producing oligomers and monomers by hydrolysis reaction. Cellulose conversion of corn pericarp reached 68% (theoretical maximum yield of glucose is 63.4 g/L), and those of soybean hulls reached 65% (theoretical maximum yield of glucose is 100.14 g/L). The liquefaction process of corn pericarp needed less time than soybean hulls since the chemical composition of corn pericarp has less glucan and protein which may have led the reaction faster than soybean hulls. Even though we can obtain enough fermentable sugars from liquefaction process, an extra step is needed to separate the solid and solution by filtration because solid may act as inhibitor during fermentation. Only the hydrolysate solutions will be separated in both biomass and then ethanol fermentation will be presented with adapted yeast by comparing to wild type yeast.

TABLE 4
Composition of sugars and inhibitor in corn pericarp after liquefied.

| Component | Liquefaction time (h) | | | | | |
|---|---|---|---|---|---|---|
| (g/L) | 0 | 24 | 48 | 72 | 96 | 120 |
| Cellobiose | 0.035 | 1.686 | 3.179 | 2.839 | 3.106 | 3.194 |
| Glucose | 2.119 | 35.14 | 43.607 | 41.85 | 42.993 | 41.509 |
| Xylose | 0.772 | 1.624 | 1.591 | 1.655 | 1.789 | 1.909 |
| Arabinose | 1.696 | 2.87 | 3.471 | 3.336 | 3.53 | 3.591 |
| Acetic acid | — | 0.405 | 0.548 | 0.527 | 0.503 | 0.408 |

TABLE 5
Composition of sugars and inhibitor in soybean hull after liquefied.

| Component | Liquefaction time (h) | | | | | |
|---|---|---|---|---|---|---|
| (g/L) | 0 | 24 | 48 | 72 | 96 | 120 |
| Cellobiose | 0.038 | 4.143 | 8.003 | 11.447 | 12.765 | 12.567 |
| Glucose | 1.04 | 43.61 | 56.97 | 65.211 | 64.321 | 64.291 |
| Xylose | 1.531 | 31.736 | 30.266 | 31.128 | 31.024 | 33.768 |
| Arabinose | 1.072 | 11.751 | 13.531 | 14.101 | 15.013 | 15.709 |
| Acetic acid | — | 2.009 | 2.465 | 2.885 | 3.120 | 3.085 |

Those skilled in the art will recognize that numerous modifications can be made to the specific implementations described above. The implementations should not be limited to the particular limitations described. Other implementations may be possible. In addition, all references cited herein are indicative of the level of skill in the art and are hereby incorporated by reference in their entirety.

### REFERENCES

1. Blasi, D. A., J. Drouillard, E. C. Titgemeyer, S. I. Paisley, M. J. Brook, Soybean Hulls: Composition and Feeding Value for Beef and Dairy Cattle, Kansas State University Agricultural Extension Service, MF 2438 (January, 2000).
2. Cunha, F., T. Kreke, A. Badino, C. Farinas, E. Ximenes, and M. R. Ladisch, “Liquefaction of Sugarcane Bagasse for Enzyme Production,” Bioresource Technology, (In Press, 2014).
3. Horner, J. and J. Sexten, “By-product feed listing,” U. Missouri Extension (http://agebb.missouri.edu/dairy/byprod/bplist.asp (Dec. 18, 2014).
4. Indiana Soybean Alliance, Production Facts, (www.indianasoybean.com) (2012).
5. Kim, Y., T. Kreke, R. Hendrickson, J. Parenti, M. R. Ladisch, “Fractionation of Cellulase and Fermentation Inhibitors from Steam Pretreated Mixed Hardwood,” Bioresource Technology, 135, 30-38 (2013).
6. Kim, Y., T. Kreke, J-K. Ko, and M. R. Ladisch, “Hydrolysis-Determining Substrate Characteristics in Liquid Hot Water Pretreated Hardwood,” Biotechnol. Bioeng., DOI:10.1002/bit.25465 (In Press, 2014).
7. Kim, Y., R. Hendrickson, N. S. Mosier, M. R. Ladisch, B. Bals, V. Balan, B. E. Dale, “Enzyme Hydrolysis and Ethanol Fermentation of Liquid Hot Water and AFEX Pretreated Distillers' Grains at High-Solids Loadings,” Bioresource Technology J., 99(12), 5206-5215 (2008a).
8. Kim, Y., N. Mosier, M. R. Ladisch, “Process Simulation of Modified Dry Grind Ethanol Plant with Recycle of Pretreated and Enzymatically Hydrolyzed Distillers' Grains,” Bioresource Technology J., 99(12), 5177-5192 (2008b).
9. Ko, J-K., E. Ximenes, Y. Kim, and M. R. Ladisch, “Adsorption of Enzyme onto Lignins of Liquid Hot Water Pretreated Hardwoods,” Biotechnol. Bioeng. (In Press, 2014a).
10. Ko, J-K., Y. Kim, E. Ximenes, and M. R. Ladisch, “Effect of Liquid Hot Water Pretreatment Severity on Properties of Hardwood Lignin and Enzymatic Hydrolysis of Cellulose,” Biotechnol. Bioeng. (In Press, 2014b).
11. Ladisch, M. R., E. Ximenes, A. S. Engelberth, and N. S. Mosier, “Biological Engineering and the Emerging Cellulose Ethanol Industry,” Advances in Developing Sustainable Sources of Biomass, New Catalysts, and Bioprocessing Technologies are Moving Lignocellulosic Biofuels Closer to Commercial Reality, CEP 110(11), 59-62 (2014).
12. Ladisch, M. R., N. S. Mosier, Y. Kim, E. Ximenes, D. Hogsett, “Converting Cellulose to Biofuels,” SBE Special Supplement Biofuels, CEP, 106(3), 56-63 (2010).
13. McAloon, A., F. Taylor, W. Yee, K. Ibsen, and R. Wooley, “Determining the Cost of Producing Ethanol from Corn Starch and Lignocellulosic Feedstocks,” Joint Study sponsored by USDA and DOE, Task BFP 1.7110 (Oct., 2000).
14. Mielenz, J. R., J. S. Bardsley, and C. E. Wyman, “Fermentation of Soybean Hulls to Ethanol While Preserving Protein Value,” Bioresource Technology, 100, 3532-3539 (2009).
15. O'Brien, D. M., The Economic Value of Soybean Meal and Hull Microbial Fermentation, Extension Agricultural Economists, Kansas State University, page 8 (gives hulls/bushel) (Aug. 10, 2011).
16. PCT VVO 2013/155341 A1, Enzyme catalyzed disassembly of corn kernels, Ladisch, M. R., Y. Kim, R. Hendrickson (Oct. 17, 2013).
17. USDA AgriNews: Indiana, Ohio Soybean Processors Report, Wednesday (Nov. 5, 2014).
18. Voegele, E., “EPA: Nearly 6 Million Cellulosic RINs Generated in November,” Ethanol Producer Magazine (Dec. 19, 2014).
19. Ximenes, E., Y. Kim, N. Mosier, B. Dien and M. Ladisch, “Deactivation of Cellulases by Phenols,” Enzyme and Microbial Technology, 48(1), 54-60 (2011).
20. Ximenes, E., Y. Kim, N. Mosier, B. Dien, and M. Ladisch, “Inhibition of Cellulases by Phenols,” Enzyme and Microbial Technology, 46(3), 170-176 (2010).
21. Zeng, M., E. Ximenes, M. R. Ladisch, N. S. Mosier, W. Vermerris, C.-P. Huang, and D. M. Sherman, “Tissue-specific Biomass Recalcitrance in Corn Stover Pretreated with Liquid Hot-water: Enzymatic Hydrolysis, Part 1,” Biotechnology and Bioengineering, 109(2), 390-397 (2012a) http://onlinelibrary.wiley.com/journal/10.1002/%28ISSN%291097-0290/homepage/media.htm.
22. Zeng, M., E. Ximenes, M. R. Ladisch, N. S. Mosier, W. Vermerris, C.-P. Huang, and D. M. Sherman, “Tissue-specific Biomass Recalcitrance in Corn Stover Pretreated with Liquid Hot-water: Enzymatic Hydrolysis, Part 2,” Biotechnology and Bioengineering, 109(2), 398-404 (2012b) (visual abstract prepared http://onlinelibrary.wiley.com/journal/10.1002/%28ISSN%291097-0290/homepage/media.htm.).

1. (canceled)

2. A method for producing a pumpable slurry from a cellulose-containing feedstock, comprising:
liquefying a cellulose-containing feedstock into a pumpable slurry, wherein the liquefying of the cellulose-containing feedstock is carried out using an enzyme in an agitated vessel.

3. (canceled)

4. The method of claim 2, wherein the feedstock has an initial average particle length in the range of about 0.1 mm to about 10 mm.

5. The method of claim 2, wherein the liquefying of the cellulose-containing feedstock is carried out between 4° C. and 100° C.

6. The method of claim 2, wherein the liquefying of the cellulose-containing feedstock is carried out above 100° C.

7. The method of claim 2, wherein the liquefying of the cellulose-containing feedstock is carried out between 4° C. and a temperature above the critical point.

8. The method of claim 2, wherein the liquefying of the cellulose-containing feedstock is carried out between 4° C. and 260° C.

9. The method of claim 5, wherein the liquefying of the cellulose-containing feedstock is carried out at a pressure of 1 bar.

10. The method of claim 2, wherein the pressure is above the pressure at which water vaporizes.

11. The method of claim 2, wherein the enzyme comprises cellulase, xylanase, protease, β-xylosidase, β-glucosidase, lipase, or any combination thereof.

12. (canceled)

13. (canceled)

14. (canceled)

15. (canceled)

16. (canceled)

17. (canceled)

18. The method of claim 2, wherein the method is carried out in a fed-batch or continuously-fed addition of the cellulose-containing feedstock.

19. (canceled)

20. The method of claim 2, wherein the cellulose-containing feedstock comprises soybean hulls, corn fiber, corn cobs, sugarcane bagasse, wheat straw, hardwood, poplar, energy crops, corn, soybean residues, switch grass, or any combination thereof.

21. (canceled)

22. (canceled)

23. (canceled)

24. (canceled)

25. (canceled)

26. (canceled)

27. (canceled)

28. (canceled)

29. (canceled)

30. (canceled)

31. The method claim 2, wherein the method results in a slurry concentration of at least 300 g/L.

32. (canceled)

33. (canceled)

34. (canceled)

35. (canceled)
